(19) United States
(12) Patent Application Publication
(10) Pub. No.: US 2024/0398765 A1
(43) Pub. Date: Dec. 5, 2024

(54) TRANSDERMAL THERAPEUTIC SYSTEM FOR THE TRANSDERMAL ADMINISTRATION OF TIZANIDINE

(71) Applicant: LTS LOHMANN THERAPIE-SYSTEME AG, Andernach (DE)

(72) Inventors: Hans-Werner WOLF, Neuwied (DE); Marco EMGENBROICH, Rheinbach (DE); Nico REUM, Mending (DE); Beatrix PLATT, Hausten (DE); Anna SCHLÜTER, Königswinter (DE); Rolf BÖHM, Kleinmaischeid (DE); Zarah BÜSCHER, Münster (DE)

(21) Appl. No.: 18/699,714

(22) PCT Filed: Oct. 14, 2022
(86) PCT No.: PCT/EP2022/078678

§ 371 Date: Apr. 9, 2024

(30) Foreign Application Priority Data
Oct. 15, 2021 (EP) 21202959.9

(51) Int. Cl. A61K 31/433; A61K 9/70; A61K 47/12; A61K 47/32; A61K 47/34

(52) U.S. Cl. CPC A61K 31/433; A61K 9/7084; A61K 47/12; A61K 47/32; A61K 47/34

(57) ABSTRACT

The present invention relates to transdermal therapeutic systems (TTS) for the transdermal administration of tizanidine.

### TECHNICAL FIELD OF THE INVENTION

The present invention relates to a transdermal therapeutic system (TTS) for the transdermal administration of tizanidine, processes of manufacture, and uses thereof.

### BACKGROUND OF THE INVENTION

The active ingredient tizanidine (also known as e.g. [5-chloro-4-(2-imidazolin-2-ylamino)-2,1,3-benzothiadiazole] or CAS No. 51322-75-9), is a centrally-acting skeletal muscle relaxant. It is an α₂-adrenergic agonist, acting mainly at spinal and supraspinal levels to inhibit excitatory interneurones. Tizanidine in the form of its free base has the following chemical formula.

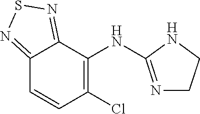 [SMILES: Clc1ccc2nsnc2c1NC1=NCCN1]

Tizanidine in the form of its free base or tizanidine in the form of its hydrochloride is used for the symptomatic relief of spasticity associated with multiple sclerosis or with spinal cord injury, and in the symptomatic treatment of painful muscle spasm.

Tizanidine can be administered via the oral route or via the transdermal route. However, there is currently no commercial TTS for the transdermal administration of tizanidine.

One challenge in developing a TTS for the transdermal administration of tizanidine is that tizanidine in the form of its hydrochloride does not permeate through the skin, whereas tizanidine in the form of its free base slightly permeates through the skin. This challenge was e.g. addressed in US 2018/0236082 A1 by developing a complex TTS in which tizanidine in the form of its hydrochloride is converted into tizanidine in the form of its free base. Skin permeability was achieved with such TTS. However, the formulation of this TTS includes more than 15 components and is therefore rather complex. In addition, the adhesiveness of the tizanidine-containing layer of this TTS is not satisfactory and it is not possible to produce such complex TTS in a constant quality, which is necessary for a large-scale production. In addition, a storage stability of such TTS is not sufficient.

There is a need in the art for a TTS for the transdermal administration of tizanidine that does not have the aforementioned problems.

### OBJECTS AND SUMMARY OF THE INVENTION

It is an object of certain embodiments of the present invention to provide a TTS for the transdermal administration of tizanidine in the form of its free base.

It is a further object of certain embodiments of the present invention to provide a TTS for the transdermal delivery of tizanidine in the form of its free base, wherein said TTS has a less complex structure, e.g. including less than 15, less than 10 or less than 6 components for said transdermal delivery.

It is a further object of certain embodiments of the present invention to provide a TTS for the transdermal delivery of tizanidine in the form of its free base, wherein said TTS can be produced in a constant quality.

It is a further object of certain embodiments of the present invention to provide a TTS for the transdermal administration of tizanidine in the form of its free base, which provides a permeation rate, which is sufficient for achieving a therapeutically effective dose over an administration period, such as for at least 72 hours.

It is a further object of certain embodiments of the present invention to provide a TTS for the transdermal administration of tizanidine in the form of its free base, which provides an improved permeation rate over an administration period, such as for at least 20 hours.

It is a further object of the present invention to provide a TTS for the transdermal administration of tizanidine in the form of its free base with a high active-agent utilization, i.e. a TTS, which does not require a high excess amount of active agent to provide a sufficient release performance during an administration period.

It is a further object of certain embodiments of the present invention to provide a TTS for the transdermal administration of tizanidine in the form of its free base, which is suitable use in a method of treating a human patient.

It is an object of certain embodiments of the present invention to provide a TTS for the transdermal administration of tizanidine in the form of its free base, wherein said TTS is easy to manufacture.

These objects and others are accomplished by the present invention which, according to a first aspect, is directed to a transdermal therapeutic system for the transdermal administration of tizanidine comprising a tizanidine-containing layer structure, said tizanidine-containing layer structure comprising:
A) a backing layer;
B) a tizanidine-containing layer comprising:
   1. a therapeutically effective amount of tizanidine in the form of its free base;
   2. at least one polar polymer;
   3. at least one nonpolar polymer; and
   4. at least one fatty acid.

According to certain embodiments, B) the tizanidine-containing layer further comprises
5. an additive,
wherein said additive is selected from the group consisting of lauryl lactate, methyl laurate, dihydrolevoglucosenone, dimethyl propylene urea and a combination thereof, wherein said additive is preferably lauryl lactate; and wherein a content of the additive in the tizanidine-containing layer ranges from 1 to 20%, more preferably from 5 to 15% by weight based on the total weight of the tizanidine-containing layer and/or a mass ratio of the mass of tizanidine to the mass of the additive in the tizanidine-containing layer ranges from about 0.25 to about 4, or from about 0.3 to about 3.

According to a second aspect, the present invention is directed to a transdermal therapeutic system according to the first aspect for use in a method of treating a human patient.

### DEFINITIONS

Within the meaning of this invention, the term “transdermal therapeutic system” (TTS) refers to a system by which the active agent (e.g. tizanidine) is administered to the systemic circulation via transdermal delivery and refers to the entire individual dosing unit that is applied, after removing an optionally present release liner, to the skin of a patient, and which comprises a therapeutically effective amount of active agent in an active agent-containing layer structure and optionally an additional adhesive overlay on top of the active agent-containing layer structure. The active agent-containing layer structure may be located on a release liner (a detachable protective layer), thus, the TTS may further comprise a release liner. Within the meaning of this invention, the term “TTS” in particular refers to systems providing transdermal delivery, excluding active delivery for example via iontophoresis or microporation. Transdermal therapeutic systems may also be referred to as transdermal drug delivery systems (TDDS) or transdermal delivery systems (TDS).

Within the meaning of this invention, the term “tizanidine-containing layer structure” refers to the layer structure containing a therapeutically effective amount of tizanidine and comprises a backing layer and at least one active agent-containing layer. Preferably, the tizanidine-containing layer structure is a tizanidine-containing self-adhesive layer structure.

Within the meaning of this invention, the term “therapeutically effective amount” refers to a quantity of active agent in the TTS which is, if administered by the TTS to a patient, sufficient to provide e.g. a symptomatic treatment of spasticity associated with multiple sclerosis or with spinal cord injury, and also sufficient to provide a symptomatic treatment of painful muscle spasm. Further, the TTS comprises a therapeutically effective amount, if administered to a patient, it sufficiently provides e.g. treatment of patients suffering from of chronic neck pain, lumbosacral neuralgia with a myofascial component to their pain, regional musculoskeletal pain syndromes, migraine headaches, and insomnia. The therapeutically effective amount may further be sufficient if the TTS can be used as an anticonvulsant or may be applied as part of a detoxification therapy regimen in patients exhibiting analgesic rebound headaches to assist with analgesic withdrawal. A TTS usually contains more active in the system than is in fact provided to the skin and the systemic circulation. This excess amount of active agent is usually necessary to provide enough driving force for the delivery from the TTS to the systemic circulation.

Within the meaning of this invention, the terms “active”, “active agent”, and the like, as well as the term “tizanidine” refer to tizanidine in any pharmaceutically acceptable chemical and morphological form and physical state. These forms include without limitation tizanidine in its free base/free acid form, protonated or partially protonated tizanidine, tizanidine salts, cocrystals and in particular acid/base addition salts formed by addition of an inorganic or organic acid/base such as tizanidine hydrochloride or tizanidine maleate, solvates, hydrates, clathrates, complexes and so on, as well as tizanidine in the form of particles which may be micronized, crystalline and/or amorphous, and any mixtures of the aforementioned forms. The tizanidine, where contained in a medium such as a solvent, may be dissolved or dispersed or in part dissolved and in part dispersed.

Within the meaning of this invention, the term “tizanidine in form of the free base” refers to tizanidine in any pharmaceutically acceptable chemical and morphological form and physical state. Preferably, the term does not include tizanidine in the form of tizanidine salts. In particular, the term does not include tizanidine in protonated form or in the form of tizanidine salts.

Unless otherwise indicated, in particular the amount of tizanidine in the layer structure relates to the amount of tizanidine included in the TTS during manufacture of the TTS and is calculated based on tizanidine in the form of the free base. E.g., when 0.1 mmol (equal to 25.4 mg) tizanidine base is included in the TTS during manufacture, the amount of tizanidine in the layer structure is, within the meaning of the invention, 0.1 mmol or 25.4 mg.

The tizanidine starting material included in the TTS during manufacture of the TTS may be in the form of particles. Tizanidine may e.g. be present in the active agent-containing layer structure in the form of particles and/or be dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “dispersing” refers to a step or a combination of steps wherein a starting material (e.g. tizanidine) is not totally dissolved. Dispersing in the sense of the invention comprises the dissolution of a part of the starting material (e.g. tizanidine particles), depending on the solubility of the starting material (e.g. the solubility of tizanidine in the coating composition).

There are two main types of TTS for active agent delivery, i.e. matrix-type TTS and reservoir-type TTS. The release of the active agent in a matrix-type TTS is mainly controlled by the matrix including the active agent itself. In contrast, thereto, a reservoir-type TTS typically needs a rate controlling membrane controlling the release of the active agent. In principle, also a matrix-type TTS may contain a rate-controlling membrane. However, matrix-type TTS are advantageous in that, compared to reservoir-type TTS, usually no rate determining membranes are necessary and no dose dumping can occur due to membrane rupture. In summary, matrix-type transdermal therapeutic systems (TTS) are less complex in manufacture, easy, and convenient to use by patients.

Within the meaning of this invention, “matrix-type TTS” refers to a system or structure wherein the active is homogeneously dissolved and/or dispersed within a polymeric carrier, i.e. the matrix, which forms with the active agent and optionally remaining ingredients a matrix layer. In such a system, the matrix layer controls the release of the active agent from the TTS. Preferably, the matrix layer has sufficient cohesion to be self-supporting so that no sealing between other layers is required. Accordingly, the active agent-containing layer may in one embodiment of the invention be an active agent-containing matrix layer, wherein the active agent is homogeneously distributed within a polymer matrix. In certain embodiments, the active agent-containing matrix layer may comprise two active agent-containing matrix layers, which may be laminated together. Matrix-type TTS may in particular be in the form of a “drug-in-adhesive”-type TTS referring to a system wherein the active is homogeneously dissolved and/or dispersed within a pressure-sensitive adhesive matrix. In this connection, the active agent-containing matrix layer may also be referred to as active agent-containing pressure sensitive adhesive layer or active agent-containing pressure sensitive adhesive matrix layer. A TTS comprising the active agent dissolved and/or dispersed within a polymeric gel, e.g. a hydrogel, is also considered to be of matrix-type in accordance with present invention.

Within the meaning of this invention, the term “tizanidine-containing layer” refers to a layer containing tizanidine and providing the area of release. The term covers tizanidine-containing matrix layers and tizanidine-containing reservoir layers. If the tizanidine-containing layer is a tizanidine-containing matrix layer, said layer is present in a matrix-type TTS. If the polymer is a pressure-sensitive adhesive, the matrix layer may also represent the adhesive layer of the TTS, so that no additional skin contact layer is present. Alternatively, an additional skin contact layer may be present as adhesive layer, and/or an adhesive overlay is provided. The additional skin contact layer is typically manufactured such that it is tizanidine-free. However, due to the concentration gradient, tizanidine will migrate from the matrix layer to the additional skin contact layer over time, until equilibrium is reached. The additional skin contact layer may be present on the tizanidine-containing matrix layer or separated from the tizanidine-containing matrix layer by a membrane, preferably a rate controlling membrane. Preferably, the tizanidine-containing matrix layer has sufficient adhesive properties, so that no additional skin contact layer is present. If the tizanidine-containing layer is a tizanidine-containing reservoir layer, said layer is present in a reservoir-type TTS, and the layer comprises tizanidine in a liquid reservoir. In addition, an additional skin contact layer is preferably present, in order to provide adhesive properties. Preferably, a rate-controlling membrane separates the reservoir layer from the additional skin contact layer. The additional skin contact layer can be manufactured such that it is tizanidine-free or tizanidine-containing. If the additional skin contact layer is free of active agent the active agent will migrate, due to the concentration gradient, from the reservoir layer to the skin contact layer over time, until equilibrium is reached. Additionally an adhesive overlay may be provided.

As used herein, the tizanidine-containing layer is preferably a tizanidine-containing matrix layer, and it is referred to the final solidified layer. Preferably, a tizanidine-containing matrix layer is obtained after coating and drying the solvent-containing coating composition as described herein. Alternatively a tizanidine-containing matrix layer is obtained after melt-coating and cooling. The tizanidine-containing matrix layer may also be manufactured by laminating two or more such solidified layers (e.g. dried or cooled layers) of the same composition to provide the desired area weight. The matrix layer may be self-adhesive (in the form of a pressure sensitive adhesive matrix layer), or the TTS may comprise an additional skin contact layer of a pressure sensitive adhesive for providing sufficient tack. Preferably, the matrix layer is a pressure sensitive adhesive matrix layer. Optionally, an adhesive overlay may be present.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. A pressure sensitive adhesive layer, when in contact with the skin, is “self-adhesive”, i.e. provides adhesion to the skin so that typically no further aid for fixation on the skin is needed. A “self-adhesive” layer structure includes a pressure sensitive adhesive layer for skin contact, which may be provided in the form of a pressure sensitive adhesive matrix layer or in the form of an additional layer, i.e. a pressure sensitive adhesive skin contact layer. An adhesive overlay may still be employed to advance adhesion. The pressure-sensitive adhesive properties of a pressure-sensitive adhesive depend on the polymer or polymer composition (e.g. a mixture of a silicone and an acrylic polymer; or a combination of a polyisobutylene mixture and a polyvinylpyrrolidone) are used. In particular, the pressure-sensitive adhesive according to the present invention may comprise at least one polar polymer and at least one nonpolar polymer.

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, preferably above 5000 and more preferably above 10,000 Dalton. Correspondingly, compounds with a molecular weight below 2000, preferably below 5000 or more preferably below 10,000 Dalton are usually referred to as oligomers.

Within the meaning of this invention, the term “polar polymer” is used for those polymers which preferably dissolve in, or are swollen by, water. Polar polymers are preferably essentially composed of monomer units including polar functional groups which are polar such as hydroxy-, carbonyl-, carboxyl-, amino-, quaternary ammonium-, sulfhydryl-, phosphate-, sulfate groups and certain carboxylic esters. The term polar polymers encompasses polar acrylic polymers and polyvinylpyrrolidones.

Within the meaning of this invention, the term “nonpolar polymer” is used for those polymers which neither dissolve in, nor are swollen by, water. Nonpolar polymers are essentially composed of monomers which are nonpolar. Nonpolar polymers encompass materials such as polyethylene, polyisobutylene, polystyrene, polyvinylchloride, polytetrafluorethylene, or polysiloxanes.

Within the meaning of this invention, the term “skin contact layer” refers to the layer included in the active agent-containing layer structure to be in direct contact with the skin of the patient during administration. This may be the active agent-containing layer. When the TTS comprises an additional skin contact layer, the other layers of the active agent-containing layer structure do not contact the skin and do not necessarily have self-adhesive properties. As outlined above, an additional skin contact layer attached to the active agent-containing layer may over time absorb parts of the active agent. An additional skin contact layer may be used to enhance adherence. The sizes of an additional skin contact layer and the active agent-containing layer are usually coextensive and correspond to the area of release. However, the area of the additional skin contact layer may also be greater than the area of the active agent-containing layer. In such a case, the area of release still refers to the area of the active agent-containing layer.

Within the meaning of this invention, the term “area weight” refers to the dry weight of a specific layer, e.g. of the matrix layer, provided in g/m². The area weight values are subject to a tolerance of ±10%, preferably ±7.5%, due to manufacturing variability.

If not indicated otherwise “%” refers to % by weight.

Within the meaning of this invention, the term “cross-linking agent” refers to a substance which is able to cross-link functional groups contained within the polymer.

Within the meaning of this invention, the term “adhesive overlay” refers to a self-adhesive layer structure that is free of active agent and larger in area than the active agent-containing structure and provides additional area adhering to the skin, but no area of release of the active agent. It enhances thereby the overall adhesive properties of the TTS. The adhesive overlay comprises a backing layer that may provide occlusive or non-occlusive properties and an adhesive layer. Preferably, the backing layer of the adhesive overlay provides non-occlusive properties.

Within the meaning of this invention, the term “backing layer” refers to a layer which supports the active agent-containing layer or forms the backing of the adhesive overlay. At least one backing layer in the TTS and usually the backing layer of the active agent-containing layer is substantially impermeable to the active agent contained in the layer during the period of storage and administration and thus prevents active loss or cross-contamination in accordance with regulatory requirements. Preferably, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyurethanes, and mixtures thereof. Suitable backing layers are thus for example PET laminates, EVA-PET laminates and PE-PET laminates. Also suitable are woven or non-woven backing materials.

The TTS according to the present invention can be characterized by certain parameters as measured in an in vitro skin permeation test.

The in vitro permeation test is performed in a Franz diffusion cell with dermatomed split-thickness human skin with a thickness of 500 μm and an intact epidermis, and with phosphate buffer pH 5.5 as receptor medium (32° C. with 0.1% saline azide).

The amount of active permeated into the receptor medium is determined in regular intervals using a validated HPLC method with a UV photometric detector by taking a sample volume. The receptor medium is completely or in part replaced by fresh medium when taking the sample volume, and the measured amount of active permeated relates to the amount permeated between the two last sampling points and not the total amount permeated so far.

The permeated amount is given as a “cumulative permeated amount”, corresponding to the cumulated amount of active permeated at a certain point in time. E.g., in an in vitro permeation test as described above, wherein the amount of active permeated into the receptor medium has been e.g. measured at hours 0, 3, 6, 8, 24 and 32, the “cumulative permeated amount” of active at hour 32 corresponds to the sum of the permeated amounts from hour 0 to hour 3, hour 3 to hour 6, hour 6 to hour 8, hour 8 to 24 and hour 24 to hour 32.

Within the meaning of this invention, the above parameter “cumulative permeated amount” refers to mean values calculated from at least 3 in vitro permeation test experiments. Where not otherwise indicated, the standard deviation (SD) of these mean values refer to a corrected sample standard deviation, calculated using the formula:

$$SD = \sqrt{\frac{1}{n - 1} {\sum }_{i = 1}^{n} {( {x}_{i} - \overline{x} )}^{2}}$$

wherein n is the sample size, {X₁, X₂, . . . . Xₙ} are the observed values and x is the mean value of the observed values.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “dissolve” refers to the process of obtaining a solution, which is clear and does not contain any particles, as visible to the naked eye.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, toluene and mixtures thereof.

Within the meaning of this invention, the term “fatty acid” refers to any carboxylic acid with a long aliphatic chain, which is either saturated or unsaturated. In particular, a fatty acid has an unbranched chain of an even number of carbon atoms, ranging from about 4 to about 28.

Within the meaning of this invention, the term “additive” refers to any component of a tizanidine-containing layer within a TTS according to the present invention besides tizanidine in the form of its free base, at least one polar polymer, at least one nonpolar polymer and a fatty acid.

### BRIEF DESCRIPTION OF THE DRAWINGS

FIG. 1 depicts the cumulative permeated amount of tizanidine for TTS prepared according to Example 1 (Ex. 1), and Comparative Example 1A (Comp. 1A) over a time interval of 32 hours.

FIG. 2 depicts the cumulative permeated amount of tizanidine for TTS prepared according to Example 4 (Ex. 4), and Comparative Example 1A (Comp. 1A) over a time interval of 32 hours.

FIG. 3 depicts the skin permeation rate of tizanidine for TTS prepared according to Examples 2 (Ex. 2) & Example 3 (Ex. 3) and Comparative Example 1A (Comp. 1A) over a time interval of 32 hours.

### DETAILED DESCRIPTION

### TTS Structure

The present invention relates to a transdermal therapeutic system for the transdermal administration of tizanidine comprising a tizanidine-containing layer structure.

The tizanidine-containing layer structure according to the invention comprises A) a backing layer and B) a tizanidine-containing layer comprising a therapeutically effective amount of tizanidine in the form of its free base, at least one polar polymer, at least one nonpolar polymer and at least one fatty acid. The tizanidine-containing layer structure is preferably a tizanidine-containing self-adhesive layer structure.

The backing layer is preferably substantially tizanidine-impermeable. Furthermore, it is preferred that the backing layer is occlusive as outlined above.

The tizanidine-containing layer may be directly attached to the backing layer, so that no further layer between the backing layer and the tizanidine-containing layer is present.

The TTS according to the present invention may be a matrix-type TTS or a reservoir-type TTS, and preferably is a matrix-type TTS.

The tizanidine-containing layer structure according to the invention is normally located on a detachable protective layer (release liner), from which it is removed immediately before application to the surface of the patient's skin. Thus, the TTS may further comprise a release liner. A TTS protected this way is usually stored in a blister pack or a scam-sealed pouch. The packaging may be child resistant and/or senior friendly.

In a preferred embodiment of the present invention, the tizanidine-containing layer is a tizanidine-containing pressure sensitive adhesive layer and represents the skin contact layer. That is, the tizanidine-containing layer structure does not comprise an additional skin contact layer attached to the tizanidine-containing layer. In this connection, the tizanidine-containing layer is preferably a tizanidine-containing matrix layer, which is self-adhesive. The self-adhesive properties of the tizanidine-containing layer structure are preferably provided by the at least one polar polymer and/or the at least nonpolar polymer. Thus, in a preferred embodiment of the invention, the at least one polar polymer and/or the at least one nonpolar polymer is a pressure sensitive adhesive. Further details regarding the tizanidine-containing layer and the at least one polymer according to the invention are provided further below.

In another embodiment of the present invention, the tizanidine-containing layer structure further comprises an additional skin contact layer. The skin contact layer is preferably self-adhesive and provides adhesive properties. Thus, in one embodiment of the present invention, the tizanidine-containing layer structure further comprises C) a skin contact layer on the tizanidine-containing layer. In this connection, the additional skin contact layer may also contain at least one polar polymer and/or at least one nonpolar polymer. For example, when the additional skin contact layer comprises a pressure-sensitive adhesive based on polysiloxanes and acrylic polymers, the tizanidine-containing layer may comprise the same pressure-sensitive adhesive based on polysiloxanes and acrylic polymers, or a different pressure-sensitive adhesive based on polysiloxanes and acrylic polymers or a different polymer. The additional skin contact layer is preferably obtainable by coating and drying an adhesive coating composition.

In certain embodiments of the invention, wherein the tizanidine-containing layer structure comprises an additional skin contact layer, the additional skin contact layer has an area weight of from about 10 to about 160 g/m², from about 10 to about 100 g/m², or from about 10 to about 60 g/m². The total amount of polymer contained in the skin contact layer may range from about 40% to about 100% by weight, preferably from about 50% to about 100% by weight, more preferably from about 60% to about 100% by weight based on the skin contact layer. The skin contact layer may comprise an active agent. The active agent may be tizanidine, as well. The active agent in the skin contact layer may also be an additional active agent reasonable for an administration together with tizanidine. In a preferred embodiment, the skin contact layer is free of active agent, that is, is prepared without the addition of an active agent.

According to certain embodiments of the invention, the TTS may further comprise an adhesive overlay. This adhesive overlay is in particular larger in area than the tizanidine-containing structure and is attached thereto for enhancing the adhesive properties of the overall transdermal therapeutic system. Said adhesive overlay comprises a backing layer and an adhesive layer. The adhesive overlay provides additional area adhering to the skin but does not add to the area of release of the tizanidine. The adhesive overlay comprises a self-adhesive polymer or a self-adhesive polymer mixture selected from the group consisting of silicone acrylic hybrid polymers, acrylic polymers, polysiloxanes, polyisobutylenes, and mixtures thereof, which may be identical to or different from any polymer or polymer mixture included in the tizanidine-containing layer structure. In one embodiment, the TTS is free of an adhesive overlay on top of the tizanidine-containing layer structure.

Depending on the dosage, the area of release of the TTS ranges from about 1 cm² to about 150 cm², preferably from about 5 cm² to about 130 cm², more preferably from about 10 cm² to less than 120 cm².

The TTS according to the invention may further comprise one or more anti-oxidants. The anti-oxidants may be contained in the tizanidine-containing layer or in an additional skin contact layer or in both the tizanidine-containing layer and the additional skin contact layer. Suitable anti-oxidants are sodium metabisulfite, ascorbyl palmitate, tocopherol and esters thereof, ascorbic acid, butylhydroxytoluene, butylhydroxyanisole or propyl gallate, preferably butylhydroxytoluene, ascorbyl palmitate and tocopherol. The anti-oxidants may be conveniently present in the tizanidine-containing layer, preferably in an amount of from about 0.001 to about 1.0% of the tizanidine-containing layer, more preferably in an amount of from about 0.02 to about 0.5% of the tizanidine-containing layer.

The TTS according to the invention may further comprise in addition to the above mentioned ingredients at least one excipient or further component, for example from the group of cross-linking agents, solubilizers, fillers, tackifiers, film-forming agents, plasticizers, stabilizers, softeners, substances for skincare, permeation enhancers, pH regulators, and preservatives. In general, it is preferred according to the invention that no additional excipients or additives are required. Thus, the TTS has a composition of low complexity. In certain embodiments, no further additive (e.g. a transdermal permeation enhancer) is present in the TTS.

### Tizanidine-Containing Layer

As outlined in more detail above, the TTS according to the present invention comprises a tizanidine-containing layer structure comprising a tizanidine-containing layer. The tizanidine-containing layer according to the invention comprises a therapeutically effective amount of tizanidine in the form of its free base; at least one polar polymer; at least one nonpolar polymer; and at least one fatty acid. Such composition of a tizanidine-containing layer contributes to a TTS having a less complex structure, as the tizanidine containing layer only includes a little amount of components compared to known TTS and, at the same time, provides sufficient release characteristics which are at least similar to compositions known in the art as shown in the section “release characteristics” and in the Examples section below.

According to certain embodiments, a content of tizanidine in the tizanidine-containing layer ranges from about 0.5 to about 15%, preferably from about 1 to about 12% or from about 5 to about 15%, by weight based on the total weight of the tizanidine-containing layer. It is preferred that said content of tizanidine ranges from about 1 to about 4% by weight.

Alternatively, it is preferred that said content of tizanidine ranges from about 3 to about 6% by weight. In a further alternative, it is preferred that said content of tizanidine ranges from about 9 to about 12% by weight.

According to certain embodiments, the tizanidine-containing layer further comprises an additive, wherein said additive is selected from the group consisting of lauryl lactate, methyl laurate, dihydrolevoglucosenone, dimethyl propylene urea and a combination thereof. It is preferred that said additive is lauryl lactate. It has been surprisingly found that the use of such further additive may increase the release characteristic, e.g. by providing a permeation-enhancing effect, and, at the same time, less components compared to TTS known in the art need to be used in the tizanidine-containing layer.

According to certain embodiments, a content of the additive in the tizanidine-containing layer ranges from about 1 to about 20%, more preferably from about 5 to about 15% by weight based on the total weight of the tizanidine-containing layer. Alternatively or in addition, a mass ratio of the mass of tizanidine to the mass of the additive in the tizanidine-containing layer ranges from about 0.25 to about 4, preferably from about 0.5 to about 1.0; or from about 0.3 to about 3, preferably from about 0.75 to about 1.5.

The tizanidine-containing layer may be a tizanidine-containing matrix layer or a tizanidine-containing reservoir layer. It is preferred that the tizanidine-containing layer is a tizanidine-containing matrix layer, which comprises tizanidine homogeneously dispersed or dissolved in the polymer matrix. In another preferred embodiment, the tizanidine-containing layer is a tizanidine-containing biphasic matrix layer, which comprises an inner phase comprising the therapeutically effective amount of tizanidine, and an outer phase comprising the at least one polar polymer and the at least one nonpolar polymer and the at least one fatty acid, wherein the inner phase forms dispersed deposits in the outer phase. The content of the inner phase in the biphasic matrix layer may be from about 5% to 40% by volume based on the volume of the biphasic matrix layer.

According to certain preferred embodiments, the tizanidine-containing layer is a self-adhesive tizanidine-containing matrix layer.

In a certain embodiment, the tizanidine-containing layer is obtainable by coating and drying a tizanidine-containing coating composition that comprises the tizanidine in the form of the free base, preferably by coating and drying a tizanidine-containing coating composition, which comprises the at least one polar polymer, the at least one nonpolar polymer, the at least one fatty acid and the therapeutically effective amount of tizanidine in the form of the free base.

According to certain embodiments, the area weight of the tizanidine-containing layer ranges from 50 to 220 g/m², preferably from 80 to 120 g/m². In another preferred embodiment, the tizanidine-containing layer has an area weight of from about 50 to about 200 g/m², preferably from about 60 to about 180 g/m², more preferably from about 80 to about 160 g/m², of about 100 g/m², or of about 150 g/m².

According to certain embodiments, a content of the at least one polar polymer in the tizanidine-containing layer ranges from about 10 to about 50%, preferably from about 10 to about 25% or about 10 to about 17%; or about 30 to about 45% or about 40 to about 45%, by weight based on the total weight of the tizanidine-containing layer. Preferably, the at least one polar polymer is selected from the group consisting of an acrylic polymer and a polyvinylpyrrolidone and a combination thereof. Further details regarding the at least one polar polymer according to the invention are provided further below.

According to certain embodiments, a content of the at least one nonpolar polymer in the tizanidine-containing layer ranges from about 20 to about 80%, preferably from about 30 to about 45% or from about 70 to about 80%, by weight based on the total weight of the tizanidine-containing layer. Preferably, the at least one nonpolar polymer is a silicone polymer, or a polyisobutylene, or the at least one nonpolar polymer comprises a polyisobutylene mixture. Further details regarding the at least one nonpolar polymer according to the invention are provided further below.

According to certain embodiments, a mass ratio of the mass of tizanidine to the combined mass of the at least one polar polymer and the at least one nonpolar polymer in the tizanidine-containing layer ranges from about 5×10⁻³ to about 1, preferably from about 1×10⁻² to about 8×10⁻² or from about 9.0×10⁻² to about 0.3. In this connection it is to be understood that the dry mass of the respective polymer is considered, i.e. without a solvent.

According to certain embodiments, a mass ratio of the mass of the polar polymer to the mass of the nonpolar polymer in the tizanidine-containing layer ranges from about 0.5 to about 2.0, preferably from about 0.8 to 1.2; or from about 1×10⁻² to about 4×10⁻¹, preferably from about 1.5×10⁻¹ to about 3.5×10⁻¹. In this connection it is to be understood that the dry mass of the respective polymer is considered, i.e. without a solvent.

According to certain embodiments, a mass ratio of the mass of the at least one fatty acid to the combined mass of the at least one polar polymer and the at least one nonpolar polymer in the tizanidine-containing layer ranges from about 1×10⁻² to about 4×10⁻¹. In this connection it is to be understood that the dry mass of the respective polymer is considered, i.e. without a solvent. It is further to be understood that in the event that more than one fatty acid is present in the tizanidine-containing layer, the ratio refers to the combined mass of the at least two fatty acids to the combined mass of the at least one polar polymer and the at least one nonpolar polymer.

When using an additional skin contact layer, the ingredients of the tizanidine-containing layer such as the tizanidine and optional additional active agents, optional auxiliary polymers, optional anti-oxidants, and optional additional excipients or additives may over time migrate into the additional skin contact layer. This however depends on the ingredients and the material of the skin contact layer.

In one embodiment of the present invention, the tizanidine-containing layer consists of a therapeutically active amount of tizanidine in its free base, a polar polymer, a mixture of two nonpolar polymers and at least one fatty acid. Alternatively, the tizanidine-containing layer consists of a therapeutically active amount of tizanidine in its free base, a polar polymer, a nonpolar polymer, at least one fatty acid and an additive as described herein.

### POLAR POLYMER

The tizanidine-containing layer according to the present invention comprises at least one polar polymer. The polar polymer may be selected from the group consisting of an acrylic polymer and a polyvinylpyrrolidone and a combination thereof.

In a preferred embodiment, the at least one polar polymer is a polymer-based pressure-sensitive adhesive.

According to one embodiment of the invention, the at least one polar polymer is a polymer based on acrylates i.e. an acrylic polymer, preferably a pressure-sensitive adhesive based on acrylates. Pressure-sensitive adhesives based on acrylates may also be referred to as acrylate-based pressure-sensitive adhesives, or acrylate pressure-sensitive adhesives. Pressure-sensitive adhesives based on acrylates may have a solids content preferably between 30% and 60%. Such acrylate-based pressure-sensitive adhesives may or may not comprise functional groups such as hydroxy groups, carboxylic acid groups, neutralized carboxylic acid groups and mixtures thereof. Thus, the term “functional groups” in particular refers to hydroxy-and carboxylic acid groups, and deprotonated carboxylic acid groups.

Corresponding commercial products are available e.g. from Henkel under the tradename Duro Tak®. Such acrylate-based pressure-sensitive adhesives are based on monomers selected from one or more of acrylic acid, butylacrylate, 2-ethylhexylacrylate, glycidylmethacrylate, 2-hydroxyethylacrylate, methylacrylate, methylmethacrylate, t-octylacrylamide and vinylacetate, and are provided in ethyl acetate, heptanes, n-heptane, hexane, methanol, ethanol, isopropanol, 2,4-pentanedione, toluene or xylene or mixtures thereof. Suitable acrylate-based pressure-sensitive adhesives are based on monomers selected from two or more of acrylic acid, butylacrylate, 2-ethylhexylacrylate, glycidylmethacrylate, 2-hydroxyethylacrylate, methylacrylate, methylmethacrylate, t-octylacrylamide and vinylacetate.

In one embodiment of the present invention, the at least one polar polymer may be an acrylic polymer and said acrylic polymer is a copolymer based on vinyl acetate, 2-ethylhexyl-acrylate, 2-hydroxyethyl-acrylate and optionally glycidyl-methacrylate.

Specific acrylate-based pressure-sensitive adhesives are available as:
Duro-Tak™ 387-2510 or Duro-Tak™ 87-2510 (a copolymer based on 2-ethylhexyl-acrylate, 2-hydroxyethyl-acrylate and methylacrylate, provided as a solution in ethyl acetate and hexane),
Duro-Tak™ 87-4287 (an acrylic copolymer comprising hydroxyl group, wherein the copolymer is based on vinyl acetate, 2-ethylhexyl-acrylate, and 2-hydroxyethyl-acrylate provided as a solution in ethyl acetate without cross-linking agent),
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (an acrylic copolymer comprising carboxylic acid groups, wherein the copolymer is based on acrylic acid, 2-ethylhexylacrylate, glycidylmethacrylate and methylacrylate, provided as a solution in ethyl acetate and hexane),
Duro-Tak™ 87-4098 (an acrylic copolymer comprising no functional groups, wherein the copolymer is based on 2-ethylhexyl-acrylate and vinyl acetate, provided as a solution in ethyl acetate).

In another embodiment, the at least one polar polymer may be a polyvinylpyrrolidone such as crospovidone. Preferably, the polyvinylpyrrolidone may be a soluble polyvinylpyrrolidone, but alternatively also an insoluble/cross-linked polyvinylpyrrolidone also known as crospovidones such as Kollidon® CL, Kollidon® CL-M and Kollidon® CL-SF, and polyvinylpyrrolidone-polyvinyl acetate copolymers, also known as copovidones, such as Kollidon® VA64. Further suitable polyvinylpyrrolidones may be selected from the group consisting of Plasdone™ K-12 (having a weight average molecular weight of about 3,700 to 4,300 g/mol), Plasdone™ K-17 (having a weight average molecular weight of about 9,500 to 10,500 g/mol), Plasdone™ K-25 (having a weight average molecular weight of about 33,500 to 34,500 g/mol), Plasdone™ K-29/32 (having a weight average molecular weight of about 57,500 to 58,500 g/mol), Plasdone™ K-90 (having a weight average molecular weight of about 1,299,500 to 1,300,500 g/mol), Plasdone™ C-12 (having a weight average molecular weight of about 3,700 to 4,300 g/mol), Plasdone™ C-17 (having a weight average molecular weight of about 9,500 to 10,500 g/mol), and Plasdone™ C-30 (having a weight average molecular weight of about 57,500 to 58,500 g/mol). In this connection, the average molecular weight is preferably determined via SEC-MALS.

### Nonpolar Polymer

The tizanidine-containing layer according to the present invention comprises at least one nonpolar polymer. This also means that said tizanidine-containing layer may also comprise a mixture of nonpolar polymers. Preferably, the at least one nonpolar polymer is a silicone polymer, or a polyisobutylene, or the at least one nonpolar polymer comprises a polyisobutylene mixture.

In one embodiment, said nonpolar polymer is as silicone polymer. Silicone polymers are polymers based on polysiloxanes. These polymers based on polysiloxanes are preferably pressure sensitive adhesives based on polysiloxanes. Pressure-sensitive adhesives based on polysiloxanes may also be referred to as silicone-based pressure-sensitive adhesives, or silicone pressure sensitive adhesives.

These pressure-sensitive adhesives based on polysiloxanes provide for suitable tack and for quick bonding to various skin types, including wet skin, suitable adhesive and cohesive qualities, long lasting adhesion to the skin, a high degree of flexibility, a permeability to moisture, and compatibility to many actives and film-substrates. It is possible to provide them with sufficient amine resistance and therefore enhanced stability in the presence of amines. Such pressure-sensitive adhesives are based on a resin-in-polymer concept wherein, by condensation reaction of silanol end blocked polydimethylsiloxane with a silica resin (also referred to as silicate resin), a pressure-sensitive adhesive based on polysiloxane is prepared wherein for amine stability the residual silanol functionality is additionally capped with trimethylsiloxy groups. The silanol end blocked polydimethylsiloxane content contributes to the viscous component of the visco-clastic behavior, and impacts the wetting and the spreadability properties of the adhesive. The resin acts as a tackifying and reinforcing agent, and participates in the elastic component. The correct balance between silanol end blocked polydimethylsiloxane and resin provides for the correct adhesive properties.

In view of the above, silicone-based polymers, and in particular silicone-based pressure sensitive adhesives, are generally obtainable by polycondensation of silanol endblocked polydimethylsiloxane with a silicate resin. Amine-compatible silicone-based polymers, and in particular amine-compatible silicone-based pressure sensitive adhesives, can be obtained by reacting the silicone-based polymer, in particular the silicone-based pressure sensitive adhesive, with trimethylsilyl (e.g. hexamethyldisilazane) in order to reduce the silanol content of the polymer. As a result, the residual silanol functionality is at least partly, preferably mostly or fully capped with trimethylsiloxy groups.

As indicated above, the tackiness of the silicone-based polymer may be modified by the resin-to-polymer ratio, i.e. the ratio of the silanol endblocked polydimethylsiloxane to the silicate resin, which is preferably in the range of from 70:30 to 50:50, preferably from 65:35 to 55:45. The tackiness will be increased with increasing amounts of the polydimethylsiloxane relative to the resin. High tack silicone-based polymers preferably have a resin-to-polymer ratio of 55:45, medium tack silicone-based polymers preferably have a resin-to-polymer ratio of 60:40, and low tack silicone-based polymers preferably have a resin-to-polymer ratio of 65:35. High tack silicone-based polymers preferably have a complex viscosity at 0.01 rad/s and 30° C. of about 5×10⁶ Poise, medium tack silicone-based polymers preferably have a complex viscosity at 0.01 rad/s and 30° C. of about 5×10⁷ Poise, and low tack silicone-based polymers preferably have a complex viscosity at 0.01 rad/s and 30° C. of about 5×10⁸ Poise. High tack amine-compatible silicone-based polymers preferably have a complex viscosity at 0.01 rad/s and 30° C. of about 5×10⁶ Poise, medium tack amine-compatible silicone-based polymers preferably have a complex viscosity at 0.01 rad/s and 30° C. of about 5×10⁸ Poise, and low tack amine-compatible silicone-based polymers preferably have a complex viscosity at 0.01 rad/s and 30° C. of about 5×10⁹ Poise.

Examples of silicone-based PSA compositions which are commercially available include the standard BIO-PSA series (7-4400,7-4500 and 7-4600 series), the amine compatible (endcapped) BIO-PSA series (7-4100, 7-4200 and 7-4300 series) and the Soft Skin Adhesives series (7-9800) manufactured and typically supplied in n-heptane or ethyl acetate by Dow Corning. For example, BIO-PSA 7-4201 is characterized by a solution viscosity at 25° C. and about 60% solids content in heptane of 450 mPa s and a complex viscosity at 0.01 rad/s at 30° C. of 1×108 Poise. BIO-PSA 7-4301 has a solution viscosity at 25° C. and about 60% solids content in heptane of 500 mPa s and a complex viscosity at 0.01 rad/s at 30° C. of 5×10⁶ Poise.

The pressure-sensitive adhesives based on polysiloxanes are supplied and used in solvents like n-heptane, ethyl acetate or other volatile silicone fluids. The solids content of pressure-sensitive adhesives based on polysiloxanes in solvents is usually between 60 and 85%, preferably between 70 and 80% or between 60 and 75%. The skilled person is aware that the solids content may be modified by adding a suitable amount of solvent.

Pressure-sensitive adhesives based on polysiloxanes, which are, e.g., available from Dow Corning, may be obtained according to the following scheme:

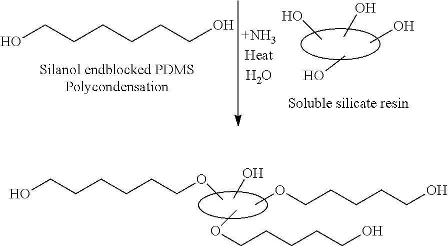

Such pressure-sensitive adhesives based on polysiloxanes are available from Dow Corning, e.g., under the tradenames BIO-PSA 7-4401, BIO-PSA-7-4501, or BIO-PSA 7-4601, which are provided in the solvent n-heptane (indicated by the code “01”), or under the tradenames BIO-PSA 7-4402, BIO-PSA 7-4502, and BIO 7-4602, which are provided in the solvent ethyl acetate (indicated by the code “02”). Typical solids contents in the solvent are in the range of from 60 to 75%. The code “44” indicates a resin-to-polymer ratio of 65:35 resulting in a low tackiness, the code “45” indicates a resin-to-polymer ratio of 60:40 resulting in medium tackiness, the code “46” indicates a resin-to-polymer ratio of 55:45 resulting in high tackiness.

Amine-compatible pressure-sensitive adhesives based on polysiloxanes, which arc, e.g., available from Dow Corning may be obtained according to the following scheme:

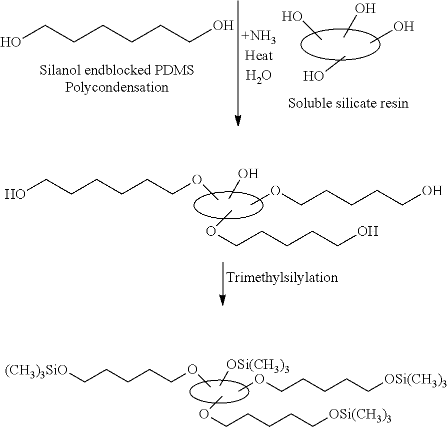

Such amine-compatible pressure-sensitive adhesives based on polysiloxanes are available from Dow Corning, e.g., under the tradenames BIO-PSA 7-4101, BIO-PSA-7-4201, or BIO-PSA 7-4301, which are provided in the solvent n-heptane (indicated by the code “01”), or under the tradenames BIO-PSA 7-4102, BIO-PSA 7-4202, and BIO 7-4302, which are provided in the solvent ethyl acetate (indicated by the code “02”). Typical solids contents in the solvent are in the range of from 60 to 75%. The code “41” indicates a resin-to-polymer ratio of 65:35 resulting in a low tackiness, the code “42” indicates a resin-to-polymer ratio of 60:40 resulting in medium tackiness, the code “43” indicates a resin-to-polymer ratio of 55:45 resulting in high tackiness. The preferred pressure-sensitive adhesives based on polysiloxanes in accordance with the invention are characterized by a solution viscosity at 25° C. and 60% solids content in n-heptane of more than about 150 mPa s, or from about 200 mPa s to about 700 mPa s, preferably as measured using a Brookfield RVT viscometer equipped with a spindle number 5 at 50 rpm. Theses may also be characterized by a complex viscosity at 0.01 rad/s at 30° C. of less than about 1×10⁹ Poise or from about 1×10⁵ to about 9×10⁸ Poise.

In a preferred embodiment, the nonpolar polymer is a silicone polymer being obtainable by polycondensation of silanol endblocked polydimethylsiloxane with a silicate resin, wherein the mass ratio of the mass of silanol endblocked polydimethylsiloxane to the mass of the silicate resin is in the range of from 70:30 to 50:50, and wherein the residual silanol functionalities of the silicone polymer are capped with trimethylsiloxy groups.

According to another embodiment, the at least one nonpolar polymer is a polyisobutylene, or the at least one nonpolar polymer comprises a polyisobutylene mixture. Preferably, the at least nonpolar polymer comprises a polyisobutylene mixture being a combination of a low molecular weight polyisobutylene and a high molecular weight polyisobutylene in a mass ratio of the mass of the low molecular weight polyisobutylene to the mass of the high molecular weight polyisobutylene ranging from 99:1 to 50:50, and wherein preferably the low molecular weight polyisobutylene has a viscosity average molecular weight of from 38,000 to 42,000 g/mol and/or a weight average molecular weight of from 34,000 to 40,000 g/mol, and wherein the high molecular weight polyisobutylene has a viscosity average molecular weight of from 1,100,000 to 1,120,000 g/mol and/or a weight average molecular weight of from 1,540,000 to 1,560,000 g/mol.

Suitable polyisobutylenes according to the invention are available under the tradename Oppanol®. Combinations of high-molecular weight polyisobutylenes (N100/N80) and low-molecular weight polyisobutylenes (B10, B11, B12, B13) may be used. Suitable ratios of low-molecular weight polyisobutylene to high-molecular weight polyisobutylene are in the range of from 100:1 to 1:100, preferably from 95:5 to 40:60, more preferably from 90:10 to 80:20. A preferred example for a polyisobutylene combination is B10/N100 in a ratio of 85/15. Oppanol® N100 has a viscosity average molecular weight Mᵥ of 1,110,000, and a weight average molecular weight M_{w} of 1,550,000, and an average molecular weight distribution M_{w}/Mₙ of 2.9. Oppanol® B10 has a viscosity average molecular weight Mᵥ of 40,000, and a weight average molecular weight M_{w} of 53,000, and an average molecular weight distribution M_{w}/Mₙ of 3.2. In certain embodiments, polybutene may be added to the polyisobutylenes. The solids content of polyisobutylenes in solvents is usually between 30 and 50%, preferably between 35 and 40%. The skilled person is aware that the solids content may be modified by adding a suitable amount of solvent.

### Fatty Acid

The tizanidine-containing layer according to the present invention comprises at least one fatty acid. Preferably, the at least one fatty acid is a saturated or unsaturated, linear or branched carboxylic acid comprising 6 to 22 carbon atoms, preferably comprising 8 to 20 carbon atoms, more preferably comprising 17 to 19 carbon atoms. Preferably, the at least one fatty acid comprises no, one, two, or three double bond(s). In a particular preferred embodiment of the present invention, the at least one fatty acid is selected from the group consisting of lauric acid, caprylic acid, oleic acid, sorbic acid, linoleic acid, levulic acid, linolenic acid, isostearic acid, and mixtures thereof, more preferably selected of the group consisting of lauric acid, caprylic acid, oleic acid, and mixtures thereof.

In certain embodiment, the tizanidine-containing layer according to the present invention comprises at least two fatty acids.

In a certain embodiment, the tizanidine-containing layer according to the present invention comprises oleic acid and optionally at least one further fatty acid. In this connection, it is preferred that the tizanidine-containing layer according to the present invention comprises oleic acid in an amount of about 5 to about 20%, preferably of about 7 to about 15%, and in particular of about 7 to about 13%, by weight based on the total weight of the tizanidine-containing layer.

In a certain embodiment, the content of the at least one fatty acid in the tizanidine-containing layer ranges from 5 to 25%, preferably from 5 to 20%, and in particular from 10 to 20%, by weight based on the total weight of the tizanidine-containing layer. In a further embodiment, the mass ratio of the mass of the at least one fatty acid to the combined mass of the of the at least one polar polymer and the at least one nonpolar polymer in the tizanidine-containing layer ranges from about 1×10⁻² to about 4×10⁻¹.

### Release Characteristics

The TTS in accordance with the invention are designed for transdermally administering tizanidine to the systemic circulation for a predefined extended period of time (such as about 32 hours).

In a certain embodiment, the TTS according to the present invention provides a cumulative permeated amount of tizanidine as measured in a Franz diffusion cell with dermatomed human skin of 86 μg/cm² to 150 μg/cm², preferably of 100 μg/cm² to 120 μg/cm² over a time period of 32 hours.

According to certain embodiments, the TTS according to the present invention provides a cumulative permeated amount of tizanidine as measured in a Franz diffusion cell with dermatomed human skin of 148 μg/cm² to 200 μg/cm², preferably of 151 μg/cm² to 160 g/cm² over a time period of 32 hours.

According to certain embodiments, the TTS according to the present invention provides a cumulative permeated amount of tizanidine as measured in a Franz diffusion cell with dermatomed human skin of 295 μg/cm² to 750 μg/cm², preferably of 500 μg/cm² to 650 μg/cm² or 500 to 600 μg/cm² over a time period of 32 hours.

In further certain embodiments, the TTS according to the present invention provides a cumulative permeated amount of tizanidine as measured in a Franz diffusion cell with dermatomed human skin of 55 μg/cm² to 100 μg/cm², preferably of 65 μg/cm² to 85 μg/cm² over a time period of 24 hours.

According to certain embodiments, the TTS according to the present invention provides a cumulative permeated amount of tizanidine as measured in a Franz diffusion cell with dermatomed human skin of 80 μg/cm² to 140 μg/cm², preferably of 100 μg/cm² to 130 μg/cm² over a time period of 24 hours.

According to certain embodiments, the TTS according to the present invention provides a cumulative permeated amount of tizanidine as measured in a Franz diffusion cell with dermatomed human skin of 200 μg/cm² to 700 μg/cm², preferably of 300 μg/cm² to 650 μg/cm² or 400 to 600 μg/cm² over a time period of 24 hours.

In further certain embodiments, the TTS according to the present invention provides tizanidine as measured in a Franz diffusion cell with dermatomed human skin in an amount of 2 mg to 48 mg, preferably of 4 mg to mg over a time period of 24 hours.

### Medical Use/Method of Treatment

In accordance with a second aspect of the present invention, the TTS according to the present invention is for use in a method of treating a human patient. Such method of treating a human patient may comprise administering the TTS on the skin of the patient. It is preferred that the TTS according to the present invention is for use in a method of treatment of spasticity associated with multiple sclerosis or spasticity associated with spinal cord injury in a human patient. In particular, such treatment comprises a step of administering the TTS to the skin of a human patient. It is further preferred that the TTS according to the present invention is for use in a method of treatment of patients suffering from chronic neck pain, lumbosacral neuralgia with a myofascial component to their pain, regional musculoskeletal pain syndromes, migraine headaches, and insomnia. The TTS according to the present invention may further be used as an anticonvulsant or may be applied as part of a detoxification therapy regimen in patients exhibiting analgesic rebound headaches to assist with analgesic withdrawal.

According to one aspect, the invention relates to the use of a TTS according to the present invention for the manufacture of a medicament for of treating a human patient.

According to another aspect, the present invention relates to a method of treating a human patient.

### Method of Manufacture

In a certain embodiment, the method of manufacture of a tizanidine-containing layer comprises the steps of:
1) combining at least the components tizanidine, the at least one fatty acid, and the polymers, in a solvent to obtain a coating composition;
2) coating the coating composition onto the backing layer or release liner or any intermediate liner; and
3) drying the coated coating composition to form the tizanidine-containing layer.

In this method of manufacture, preferably in step 1) the tizanidine is dissolved to obtain a coating composition.

In the above described method preferably the solvent is selected from alcoholic solvents, in particular methanol, ethanol, isopropanol and mixtures thereof, and from non-alcoholic solvents, in particular ethyl acetate, hexane, n-heptane, petroleum ether, toluene, and mixtures thereof, and more preferably is selected from methanol, n-heptane and ethyl acetate.

In certain embodiments, the polymer in the above method is selected from the group consisting of the herein further described polar polymer, nonpolar polymer, and mixtures thereof, which are provided as a solution and preferably as a solution in ethyl acetate, n-heptane, methanol or ethanol with a solids content of from 30 to 65% by weight.

In step 3), drying is performed preferably at a temperature of from 50 to 90° C., more 10 preferably from 60 to 80° C.

### EXAMPLES

The present invention will now be more fully described with reference to the accompanying examples. It should be understood, however, that the following description is illustrative only and should not be taken in any way as a restriction of the invention.

### Example 1

### Coating Composition

The formulations of the tizanidine base-containing coating compositions are summarized below.

TABLE 1

Example 1

| | Amt | Solids* | Solids |
|---|---|---|---|
| Ingredient (Trade Name) | [g] | [g] | [%] |
| Tizanidine base | 2.01 | 2.0 | 5.0 |
| Polyacrylate adhesive | 42.77 | 16.7 | 41.4 |
| in ethyl acetate | | | |
| Solids content of 39% | | | |
| by weight (DURO-TAK ™ | | | |
| 87-4287 from Henkel) | | | |
| Polysiloxane adhesive in | 26.72 | 16.6 | 41.2 |
| ethyl acetate. Solids content | | | |
| of 62% by weight (DOW | | | |
| CORNING ® Bio-PSA 7-4202) | | | |
| Oleic acid | 3.00 | 3.0 | 7.4 |
| Lauric acid | 2.00 | 2.0 | 5.0 |
| Methanol | 3.08 | | |
| Ethyl acetate | 1.18 | | |
| Total | 80.76 | 40.3 | 100 |

| | |
|---|---|
| Area Weight [g/m²] | 100.0 |
| Loading API [μg/cm²] | 500 |

*weight without solvent (dry weight)

### Preparation of the Coating Composition

A beaker was loaded with the tizanidine, methanol and oleic acid. Lauric acid, the acrylic pressure sensitive adhesive Duro Tak™ 87-4287, polysiloxane adhesive Bio-PSA 7-4202and the solvent (ethyl acetate) was added and the mixture was then stirred at up to 600 rpm until a homogeneous mixture was obtained.

### Coating of the Coating Composition

The resulting tizanidine-containing coating composition was coated on a polyester film (fluoro polymer coated, 75 μm thickness, which may function as release liner) and dried for approx. 10 min at room temperature and 10 min at 80° C. The coating thickness gave an area weight of the matrix layer of 100 g/m². The dried film was laminated with a polyethylene terephthalate backing layer (23 μm thickness) to provide a tizanidine-containing self-adhesive layer structure.

### Preparation of the TTS (All Examples)

The individual systems (TTS) were then punched out from the tizanidine-containing self-adhesive layer structure.

In specific embodiments a TTS as described above can be provided with an adhesive overlay, i.e. a further self-adhesive layer structure of larger surface area, preferably with rounded corners, comprising a pressure-sensitive adhesive matrix layer which is free of active ingredient and a preferably skin-colored backing layer. The TTSs are then punched out and sealed into pouches of the primary packaging material.

### Example 2

### Coating Composition

The formulations of the tizanidine base-containing coating compositions are summarized below.

TABLE 2

Example 2

| | Amt | Solids* | Solids |
|---|---|---|---|
| Ingredient (Trade Name) | [g] | [g] | [%] |
| Tizanidine base | 1.50 | 1.5 | 9.9 |
| Polyacrylate adhesive | 13.71 | 5.3 | 35.1 |
| in ethyl acetate | | | |
| Solids content of 39% | | | |
| by weight (DURO-TAK ™ | | | |
| 87-4287 from Henkel) | | | |
| Polysiloxane adhesive in | 8.58 | 5.3 | 35.1 |
| ethyl acetate. Solids content | | | |
| of 62% by weight (DOW | | | |
| CORNING ® Bio-PSA 7-4202) | | | |
| Oleic acid | 1.49 | 1.5 | 9.9 |
| Caprylic acid | 1.52 | 1.5 | 9.9 |
| Methanol | 2.04 | | |
| Total | 28.84 | 15.1 | 100 |

| | |
|---|---|
| Area Weight [g/m²] | 100.0 |
| Loading API [μg/cm²] | 1000.0 |

*weight without solvent (dry weight)

### Preparation of the Coating Composition

A beaker was loaded with the tizanidine and with the oleic acid, with caprylic acid and methanol. The mixture was stirred until dissolution of the tizanidine. The Acrylic pressure sensitive adhesive Duro Tak™ 87-4287 and the polysiloxane adhesive Bio-PSA 7-4202 was added. The mixture was then stirred at up to 600 rpm until a homogeneous mixture was obtained.

### Coating of the Coating Composition

See Example 1.

### Preparation of the TTS (All Examples)

See Example 1.

### Example 3

### Coating Composition

The formulations of the tizanidine base-containing coating compositions are summarized below.

TABLE 3

Example 3

| | Amt | Solids* | Solids |
|---|---|---|---|
| Ingredient (Trade Name) | [g] | [g] | [%] |
| Tizanidine base | 1.49 | 1.5 | 9.9 |
| Polyacrylate adhesive | 13.77 | 5.4 | 35.5 |
| in ethyl acetate | | | |
| Solids content of 39% | | | |
| by weight (DURO-TAK ™ | | | |
| 87-4287 from Henkel) | | | |
| Polysiloxane adhesive in | 8.53 | 5.3 | 34.9 |
| ethyl acetate. Solids content | | | |
| of 62% by weight (DOW | | | |
| CORNING ® Bio-PSA 7-4202) | | | |
| Oleic acid | 1.50 | 1.5 | 9.9 |
| Lauryl lactate | 1.51 | 1.5 | 9.9 |
| Methanol | 5.10 | | |
| Total | 31.90 | 15.2 | 100 |

| | |
|---|---|
| Area Weight [g/m²] | 100.0 |
| Loading API [μg/cm²] | 1000.0 |

*weight without solvent (dry weight)

### Preparation of the Coating Composition

A beaker was loaded with oleic acid, lauryl lactate and methanol. The tizanidine was added under stirring up to 600 rpm. The acrylic pressure sensitive adhesive Duro Tak™ 87-4287 and with the polysiloxane adhesive Bio-PSA 7-4202were added. The mixture was then stirred at up to 600 rpm until a homogeneous mixture was obtained.

### Coating of the Coating Composition

See Example 1.

### Preparation of the TTS (All Examples)

See Example 1.

### Example 4

Coating Composition

The formulations of the tizanidine base-containing coating compositions are summarized below.

TABLE 4

| | Example 4 | | |
|---|---|---|---|
| | Amt | Solids* | Solids |
| Ingredient (Trade Name) | [g] | [g] | [%] |
| Tizanidine base | 0.36 | 0.36 | 2.0 |
| Crospovidone | 2.70 | 2.70 | 15.0 |
| (polyvinylpyrrolidone having a | | | |
| bulk density of 0.3 to 0.4 g/ml; | | | |
| Kollidon ® CL from BASF) | | | |
| Pressure-sensitive adhesive | 33.69 | 13.1 | 72.9 |
| based on polyisobutylenes | | | |
| in n-heptane/n-hexane | | | |
| Solids content of 39% by | | | |
| weight (Oppanol ™ B10/N100 in | | | |
| a ratio of 85/15** from BASF) | | | |
| Oleic acid | 1.81 | 1.8 | 10.0 |
| n-Heptane | 1.96 | | |
| Propylene glycol | 5.19 | | |
| Total | 45.71 | 17.96 | 100 |

| | |
|---|---|
| Area Weight [g/m²] | 100.0 |
| Loading API [μg/cm²] | 270 |

*weight without solvent (dry weight);
**denotes the dry weight ratio of Oppanol ™ B10/N100

### Preparation of the Coating Composition

A beaker was loaded with the oleic acid and the solvent propylene glycol. Tizanidine was added while stirring at approx. 600 rpm and stirred until dissolution. The crospovidone (polyvinylpyrrolidone) has been micronized to a particle size d90 of about 17 to 19 μm. The micronized crospovidone, the polyisobutylene pressure sensitive adhesive and the n-heptane was added. The mixture was then stirred from approx. 600 rpm until a homogenous mixture was obtained.

### Coating of the Coating Composition See Example 1.

### Preparation of the TTS (All Examples)

See Example 1.

### Comparative Example 1A

### Coating Composition

The formulations of the tizanidine base-containing coating reference compositions can be prepared according to Examples 5-5 to 5-7 of US 2018/0236082 A1 (Table 4).

The preparation of such coating composition, the coating of the composition as well as the preparation of the TTS can be carried out as described in US 2018/0236082 A1.

### Example 5

### Measurement of Skin Permeation

The permeated amount of tizanidine and the corresponding skin permeation rates of TTS prepared according to Examples 1 to 4 and Comparative Example 1A were determined by in vitro experiments in accordance with the OECD Guideline (adopted Apr. 13, 2004) carried out with a 7.0 ml Franz diffusion cell. Split thickness human skin from cosmetic surgeries (female abdomen, date of birth 1957, 1973, 1987) was used. A dermatome was used to prepare skin to a thickness of 500 μm, with an intact epidermis for all TTS. Die cuts with an area of 1.16 cm² were punched from the TTS. The permeated amount of tizanidine in the receptor medium of the Franz diffusion cell (phosphate buffer solution pH 5.5 with 0.1% saline azide as antibacteriological agent) at a temperature of 32±1° C. was measured and the corresponding cumulative permeated amount and the skin permeation rate were calculated.

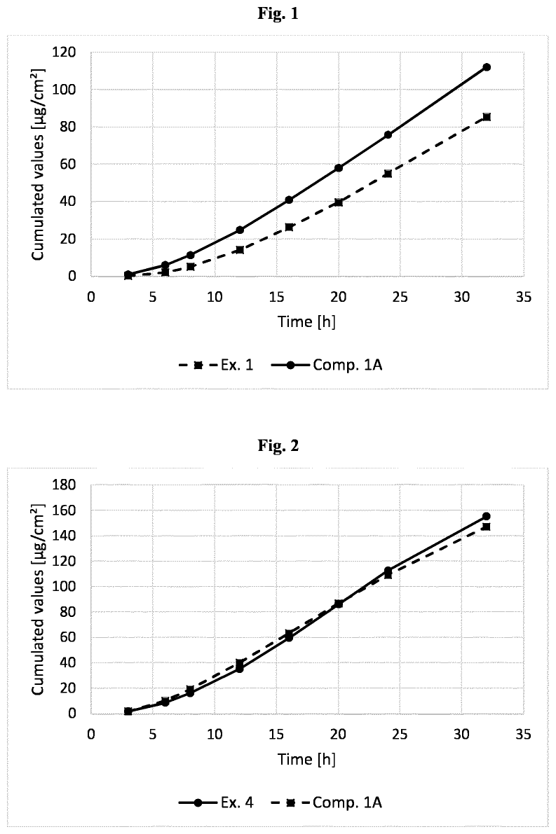

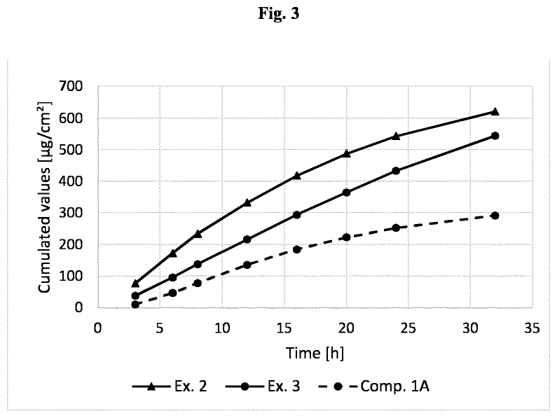

The results are shown in Table 5 below and in FIGS. 1 to 3.

TABLE 5

| | Permeation trial | | API Utilization |
|---|---|---|---|
| TTS | μg/cm² * | % of Comparative Example 1A | [%] |
| Example 1 | 112 | 131.1 | 22.4 |
| Example 2 | 620 | 213.1 | 57.9 |
| Example 3 | 544 | 186.9 | 58.5 |
| Example 4 | 155 | 105.4 | 59.2 |

* Cumulative permeated amount after 32 hours.

### The invention relates in particular to the following further items:

1. Transdermal therapeutic system for the transdermal administration of tizanidine comprising a tizanidine-containing layer structure, said tizanidine-containing layer structure comprising:
   A) a backing layer;
   B) a tizanidine-containing layer comprising:
   1. a therapeutically effective amount of tizanidine in the form of its free base;
   2. at least one polar polymer;
   3. at least one nonpolar polymer; and
   4. at least one fatty acid.
2. Transdermal therapeutic system according to item 1, wherein a content of tizanidine in the tizanidine-containing layer ranges from about 0.5 to about 15%, preferably from about 1 to about 12%, by weight based on the total weight of the tizanidine-containing layer; and/or wherein mass ratio of the mass of tizanidine to the combined mass of the at least one polar polymer and the at least one nonpolar polymer in the tizanidine-containing layer ranges from about 5×10⁻³ to about 1, preferably from about 1×10⁻² to about 8×10⁻² or from about 9.0 ×10⁻² to about 0.3.
3. Transdermal therapeutic system according to item 1 or 2, wherein the at least one polar polymer is selected from the group consisting of an acrylic polymer and a polyvinylpyrrolidone and a combination thereof.
4. Transdermal therapeutic system according to item 3, wherein the polar polymer is an acrylic polymer and said acrylic polymer is a copolymer based on vinyl acetate, 2-ethylhexyl-acrylate, 2-hydroxyethyl-acrylate and optionally glycidyl-methacrylate.
5. Transdermal therapeutic system according to item 3, wherein the polar polymer a polyvinylpyrrolidone, such as crospovidone.
6. Transdermal therapeutic system according to any one of items 1 to 5, wherein a content of the polar polymer in the tizanidine-containing layer ranges from about 10 to about 50%, preferably from about 10 to about 25% or about 30 to about 45%, by weight based on the total weight of the tizanidine-containing layer; and/or wherein a mass ratio of the mass of the polar polymer to the mass of the nonpolar polymer in the tizanidine-containing layer ranges from about 0.5 to about 2.0, or from about 1×10⁻² to about 4×10⁻¹.
7. Transdermal therapeutic system according to any one of items 1 to 6, wherein the at least one nonpolar polymer is a silicone polymer, or a polyisobutylene, or the at least one nonpolar polymer comprises a polyisobutylene mixture.
8. Transdermal therapeutic system according to item 7, wherein the nonpolar polymer is a silicone polymer being obtainable by polycondensation of silanol endblocked polydimethylsiloxane with a silicate resin, wherein the mass ratio of the mass of silanol endblocked polydimethylsiloxane to the mass of the silicate resin is in the range of from 70:30 to 50:50, and wherein the residual silanol functionalities of the silicone polymer are capped with trimethylsiloxy groups.
9. Transdermal therapeutic system according to item 7, wherein the at least nonpolar polymer comprises a polyisobutylene mixture being a combination of a low molecular weight polyisobutylene and a high molecular weight polyisobutylene in a mass ratio of the mass of the low molecular weight polyisobutylene to the mass of the high molecular weight polyisobutylene ranging from 99:1 to 50:50, and wherein preferably the low molecular weight polyisobutylene has a viscosity average molecular weight of from 38,000 to 42,000 g/mol and/or a weight average molecular weight of from 34,000 to 40,000 g/mol, and wherein the high molecular weight polyisobutylene has a viscosity average molecular weight of from 1,100,000 to 1,120,000 g/mol and/or a weight average molecular weight of from 1,540,000 to 1,560,000 g/mol.
10. Transdermal therapeutic system according to any one of items 1 to 9, wherein a content of the at least one nonpolar polymer in the tizanidine-containing layer ranges from about 20 to about 80%, preferably from about 30 to about 45% or from about 70 to about 80%, by weight based on the total weight of the tizanidine-containing layer.
11. Transdermal therapeutic system according to any one of items 1 to 10, wherein the at least one fatty acid is a saturated or unsaturated, linear or branched carboxylic acid comprising 6 to 22 carbon atoms, preferably comprising 8 to 20 carbon atoms, more preferably comprising 17 to 19 carbon atoms, or wherein the at least one fatty acid is preferably selected from the group consisting of lauric acid, caprylic acid, oleic acid, and mixtures thereof.
12. Transdermal therapeutic system according to any one of items 1 to 11, wherein a content of the at least one fatty acid in the tizanidine-containing layer ranges from 5 to 25%, preferably from 5 to 20% by weight based on the total weight of the tizanidine-containing layer; or wherein a mass ratio of the mass of the at least one fatty acid to the combined mass of the of the at least one polar polymer and the at least one nonpolar polymer in the tizanidine-containing layer ranges from about 1×10⁻² to about 4×10⁻¹.
13. Transdermal therapeutic system according to any one of items 1 to 12, wherein
   B) the tizanidine-containing layer further comprises
   5. an additive,
   wherein said additive is selected from the group consisting of lauryl lactate, methyl laurate, dihydrolevoglucosenone, dimethyl propylene urea and a combination thereof, wherein said additive is lauryl lactate; and wherein a content of the additive in the tizanidine-containing layer ranges from 1 to 20%, more preferably from 5 to 15% by weight based on the total weight of the tizanidine-containing layer; and/or a mass ratio of the mass of tizanidine to the mass of the additive in the tizanidine-containing layer ranges from about 0.25 to about 4, or from about 0.3 to about 3.
14. Transdermal therapeutic system according to any one of items 1 to 13,
wherein the tizanidine-containing layer is a tizanidine-containing matrix layer; and/or wherein the area weight of the tizanidine-containing layer ranges from 70 to 220 g/m², preferably from 80 to 120 g/m².
15. Transdermal therapeutic system according to any one of items 1 to 14,
providing a cumulative permeated amount of tizanidine as measured in a Franz diffusion cell with dermatomed human skin of 86 μg/cm² to 150 μg/cm², preferably of 100 μg/cm² to 120 μg/cm² over a time period of 32 hours; or
providing a cumulative permeated amount of tizanidine as measured in a Franz diffusion cell with dermatomed human skin of 148 μg/cm² to 200 μg/cm², preferably of 151 μg/cm² to 160 μg/cm² over a time period of 32 hours; or
providing a cumulative permeated amount of tizanidine as measured in a Franz diffusion cell with dermatomed human skin of 295 μg/cm² to 750 μg/cm², preferably of 500 μg/cm² to 650 μg/cm² or 500 to 600 μg/cm² over a time period of 32 hours.
16. Transdermal therapeutic system according to any one of the preceding items, wherein a content of tizanidine in the tizanidine-containing layer ranges from about 0.5 to about 15%, preferably from about 1 to about 12% or from about 5 to about 15%, by weight based on the total weight of the tizanidine-containing layer and wherein the at least one nonpolar polymer is a silicone polymer, or a polyisobutylene, or the at least one nonpolar polymer comprises a polyisobutylene mixture.
17. Transdermal therapeutic system according to any one of the preceding items, wherein tizanidine-containing layer structure comprises:
   A) a backing layer;
   B) a tizanidine-containing layer comprising:
   1. a therapeutically effective amount of tizanidine in the form of its free base wherein a content of tizanidine in the tizanidine-containing layer ranges from about 0.5 to about 15% by weight based on the total weight of the tizanidine-containing layer;
   2. at least one polar polymer, wherein a content of the polar polymer in the tizanidine-containing layer ranges from about 10 to about 50% based on the total weight of the tizanidine-containing layer;
   3. at least one nonpolar polymer, wherein a content of the at least one nonpolar polymer in the tizanidine-containing layer ranges from about 20 to about 80% by weight based on the total weight of the tizanidine-containing layer; and
   4. at least one fatty acid, wherein a content of the at least one fatty acid in the tizanidine-containing layer ranges from 5 to 25% by weight based on the total weight of the tizanidine-containing layer.
18. Transdermal therapeutic system according to any one of the preceding items, wherein said tizanidine-containing layer structure comprises:
   A) a backing layer;
   B) a tizanidine-containing layer comprising:
   1. a therapeutically effective amount of tizanidine in the form of its free base wherein a content of tizanidine in the tizanidine-containing layer ranges from about 0.5 to about 15% by weight based on the total weight of the tizanidine-containing layer;
   2. at least one polar polymer, wherein a content of the polar polymer in the tizanidine-containing layer ranges from about 10 to about 50% based on the total weight of the tizanidine-containing layer;
   3. at least one nonpolar polymer, wherein a content of the at least one nonpolar polymer in the tizanidine-containing layer ranges from about 20 to about 80% by weight based on the total weight of the tizanidine-containing layer;
   4. at least one fatty acid, wherein a content of the at least one fatty acid in the tizanidine-containing layer ranges from 5 to 25% by weight based on the total weight of the tizanidine-containing layer; and
   5. an additive, wherein a content of the additive in the tizanidine-containing layer ranges from about 1 to about 20% by weight based on the total weight of the tizanidine-containing layer.
19. Transdermal therapeutic system according to any one of the preceding items, wherein said tizanidine-containing layer structure comprises:
   A) a backing layer;
   B) a tizanidine-containing layer comprising:
   1. a therapeutically effective amount of tizanidine in the form of its free base;
   2. at least one polar polymer being a polyvinylpyrrolidone;
   3. at least one nonpolar polymer, wherein the at least one nonpolar polymer comprises a polyisobutylene mixture; and
   4. at least one fatty acid being oleic acid.
20. Transdermal therapeutic system according to item 19, wherein said tizanidine-containing layer structure comprises:
   A) a backing layer;
   B) a tizanidine-containing layer consists of:
   1. a therapeutically effective amount of tizanidine in the form of its free base;
   2. a polyvinylpyrrolidone;
   3. a polyisobutylene mixture; and
   4. at least one fatty acid being oleic acid.
21. Transdermal therapeutic system according to any of items 1 to 18, wherein said tizanidine-containing layer structure comprises:
   A) a backing layer;
   B) a tizanidine-containing layer comprising:
   1. a therapeutically effective amount of tizanidine in the form of its free base;
   2. at least one polar polymer being an acrylic polymer;
   3. at least one nonpolar polymer being a silicone polymer; and
   4. at least one fatty acid being a mixture of oleic acid and lauric acid.
22. Transdermal therapeutic system according to item 21, wherein said tizanidine-containing layer structure comprises:
   A) a backing layer;
   B) a tizanidine-containing layer consisting of:
   1. a therapeutically effective amount of tizanidine in the form of its free base;
   2. at least one polar polymer being an acrylic polymer;
   3. at least one nonpolar polymer being a silicone polymer; and
   4. at least one fatty acid being a mixture of oleic acid and lauric acid.
23. Transdermal therapeutic system according to any of items 1 to 18, wherein said tizanidine-containing layer structure comprises:
   A) a backing layer;
   B) a tizanidine-containing layer comprising:
   1. a therapeutically effective amount of tizanidine in the form of its free base;
   2. at least one polar polymer being an acrylic polymer;
   3. at least one nonpolar polymer being a silicone polymer; and
   4. at least one fatty acid being a mixture of oleic acid and caprylic acid.
24. Transdermal therapeutic system according to any item, wherein said tizanidine-containing layer structure comprises:
   A) a backing layer;
   B) a tizanidine-containing layer consisting of:
   1. a therapeutically effective amount of tizanidine in the form of its free base;
   2. at least one polar polymer being an acrylic polymer;
   3. at least one nonpolar polymer being a silicone polymer; and
   4. at least one fatty acid being a mixture of oleic acid and caprylic acid.
25. Transdermal therapeutic system according to any of items 1 to 18, wherein said tizanidine-containing layer structure comprises:
   A) a backing layer;
   B) a tizanidine-containing layer comprising:
   1. a therapeutically effective amount of tizanidine in the form of its free base;
   2. at least one polar polymer being an acrylic polymer;
   3. at least one nonpolar polymer being a silicone polymer; and
   4. at least one fatty acid being oleic acid; and
   5. an additive, wherein said additive is lauryl lactate.
26. Transdermal therapeutic system according to any item, wherein said tizanidine-containing layer structure comprises:
   A) a backing layer;
   B) a tizanidine-containing layer consisting of:
   1. a therapeutically effective amount of tizanidine in the form of its free base;
   2. at least one polar polymer being an acrylic polymer;
   3. at least one nonpolar polymer being a silicone polymer; and
   4. at least one fatty acid being oleic acid; and
   5. an additive, wherein said additive is lauryl lactate.
27. Transdermal therapeutic system according to any one of the preceding items, wherein the tizanidine-containing layer is a self-adhesive layer.
28. Transdermal therapeutic system according to any one of the preceding items, wherein the tizanidine-containing layer is a tizanidine-containing matrix layer.
29. Transdermal therapeutic system according to any one of the preceding items, wherein the tizanidine-containing layer is a tizanidine-containing reservoir layer.
30. Transdermal therapeutic system according to any one of the preceding items for use in a method of treating a human patient.
31. Transdermal therapeutic system according to any one of items 1 to 30 for use in a method of treating of spasticity associated with multiple sclerosis or spasticity associated with spinal cord injury in a human patient.

1. Transdermal therapeutic system for the transdermal administration of tizanidine comprising a tizanidine-containing layer structure, said tizanidine-containing layer structure comprising:
A) a backing layer;
B) a tizanidine-containing layer comprising:
1. a therapeutically effective amount of tizanidine in the form of its free base;
2. at least one polar polymer;
3. at least one nonpolar polymer; and
4. at least one fatty acid.

2. Transdermal therapeutic system according to claim 1, wherein a content of tizanidine in the tizanidine-containing layer ranges from about 0.5 to about 15%, preferably from about 1 to about 12%, by weight based on the total weight of the tizanidine-containing layer; and/or wherein mass ratio of the mass of tizanidine to the combined mass of the at least one polar polymer and the at least one nonpolar polymer in the tizanidine-containing layer ranges from about 5.0×10⁻³ to about 1, preferably from about 1×10⁻² to about 8×10⁻² or from about 9.0×10⁻² to about 0.3.

3. Transdermal therapeutic system according to claim 1 or 2, wherein the at least one polar polymer is selected from the group consisting of an acrylic polymer and a polyvinylpyrrolidone and a combination thereof.

4. Transdermal therapeutic system according to claim 3, wherein the polar polymer is an acrylic polymer and said acrylic polymer is a copolymer based on vinyl acetate, 2-ethylhexyl-acrylate, 2-hydroxyethyl-acrylate and optionally glycidyl-methacrylate.

5. Transdermal therapeutic system according to claim 3, wherein the polar polymer a polyvinylpyrrolidone, such as crospovidone.

6. Transdermal therapeutic system according to any one of claims 1 to 5, wherein a content of the polar polymer in the tizanidine-containing layer ranges from about 10 to about 50%, preferably from about 10 to about 25% or about 30 to about 45%, by weight based on the total weight of the tizanidine-containing layer; and/or wherein a mass ratio of the mass of the polar polymer to the mass of the nonpolar polymer in the tizanidine-containing layer ranges from about 0.5 to about 2.0, or from about 1×10⁻² to about 4×10⁻¹.

7. Transdermal therapeutic system according to any one of claims 1 to 6, wherein the at least one nonpolar polymer is a silicone polymer, or a polyisobutylene, or the at least one nonpolar polymer comprises a polyisobutylene mixture, preferably wherein a content of tizanidine in the tizanidine-containing layer ranges from about 0.5 to about 15%, more preferably from about 1 to about 12% or from about 5 to about 15%, by weight based on the total weight of the tizanidine-containing layer.

8. Transdermal therapeutic system according to claim 7, wherein the nonpolar polymer is a silicone polymer being obtainable by polycondensation of silanol endblocked polydimethylsiloxane with a silicate resin, wherein the mass ratio of the mass of silanol endblocked polydimethylsiloxane to the mass of the silicate resin is in the range of from 70:30 to 50:50, and wherein the residual silanol functionalities of the silicone polymer are capped with trimethylsiloxy groups.

9. Transdermal therapeutic system according to claim 7, wherein the at least nonpolar polymer comprises a polyisobutylene mixture being a combination of a low molecular weight polyisobutylene and a high molecular weight polyisobutylene in a mass ratio of the mass of the low molecular weight polyisobutylene to the mass of the high molecular weight polyisobutylene ranging from 99:1 to 50:50, and wherein preferably the low molecular weight polyisobutylene has a viscosity average molecular weight of from 38,000 to 42,000 g/mol and/or a weight average molecular weight of from 34,000 to 40,000 g/mol, and wherein the high molecular weight polyisobutylene has a viscosity average molecular weight of from 1,100,000 to 1,120,000 g/mol and/or a weight average molecular weight of from 1,540,000 to 1,560,000 g/mol.

10. Transdermal therapeutic system according to any one of claims 1 to 9, wherein a content of the at least one nonpolar polymer in the tizanidine-containing layer ranges from about 20 to about 80%, preferably from about 30 to about 45% or from about 70 to about 80%, by weight based on the total weight of the tizanidine-containing layer.

11. Transdermal therapeutic system according to any one of claims 1 to 10, wherein the at least one fatty acid is a saturated or unsaturated, linear or branched carboxylic acid comprising 6 to 22 carbon atoms, preferably comprising 8 to 20 carbon atoms, more preferably comprising 17 to 19 carbon atoms, or wherein the at least one fatty acid is preferably selected from the group consisting of lauric acid, caprylic acid, oleic acid, and mixtures thereof.

12. Transdermal therapeutic system according to any one of claims 1 to 11, wherein a content of the at least one fatty acid in the tizanidine-containing layer preferably ranges from 5 to 25% more preferably from 5 to 20% by weight based on the total weight of the tizanidine-containing layer; or wherein a mass ratio of the mass of the at least one fatty acid to the combined mass of the of the at least one polar polymer and the at least one nonpolar polymer in the tizanidine-containing layer ranges from about 1×10⁻² to about 4×10⁻¹.

13. Transdermal therapeutic system according to any one of claims 1 to 12, wherein
B) the tizanidine-containing layer further comprises
5. an additive,
wherein said additive is selected from the group consisting of lauryl lactate, methyl laurate, dihydrolevoglucosenone, dimethyl propylene urea and a combination thereof, wherein said additive is preferably lauryl lactate; and wherein a content of the additive in the tizanidine-containing layer ranges from 1 to 20%, more preferably from 5 to 15% by weight based on the total weight of the tizanidine-containing layer; and/or a mass ratio of the mass of tizanidine to the mass of the additive in the tizanidine-containing layer ranges from about 0.25 to about 4, or from about 0.3 to about 3.

14. Transdermal therapeutic system according to any one of claims 1 to 13,
wherein the tizanidine-containing layer is a tizanidine-containing matrix layer; and/or wherein the area weight of the tizanidine-containing layer ranges from 70 to 220 g/m², preferably from 80 to 120 g/m².

15. Transdermal therapeutic system according to any one of claims 1 to 14,
providing a cumulative permeated amount of tizanidine as measured in a Franz diffusion cell with dermatomed human skin of 86 μg/cm² to 150 μg/cm², preferably of 100 μg/cm² to 120 μg/cm² over a time period of 32 hours; or
providing a cumulative permeated amount of tizanidine as measured in a Franz diffusion cell with dermatomed human skin of 148 μg/cm² to 200 μg/cm², preferably of 151 μg/cm² to 160 μg/cm² over a time period of 32 hours; or
providing a cumulative permeated amount of tizanidine as measured in a Franz diffusion cell with dermatomed human skin of 295 μg/cm² to 750 μg/cm², preferably of 500 μg/cm² to 650 μg/cm² or 500 to 600 μg/cm² over a time period of 32 hours.

16. Transdermal therapeutic system according to any one of claims 1 to 15 for use in a method of treating a human patient.
